(19) United States
(12) Patent Application Publication
(10) Pub. No.: US 2015/0374859 A1
(43) Pub. Date: Dec. 31, 2015

(54) Imaging compounds for tracking histone deacetylase inhibitor and synthesis method thereof

(71) Applicant: Ming-Hsin Li, Taoyuan County (TW)

(72) Inventor: Ming-Hsin Li, Taoyuan County (TW)

(21) Appl. No.: 14/315,318

(22) Filed: Jun. 25, 2014

(51) Int. Cl. A61K 51/04; C07B 59/00

(52) U.S. Cl. CPC A61K 51/0485; A61K 51/04; C07B 59/002; C07B 59/001

(57) ABSTRACT

A synthesis method of labeling histone deacetylase inhibitor (HDAC inhibitors) SC027 with Gallium-67 or Gallium-68 radionuclide, wherein a labeled precursor DOTA-SC027 which comprises DOTA-NHS-ester (1,4,7,10-tetraazacyclododecane-1,4,7,10-tetraacetic acid-N-hydroxysuccinimide-ester) and HDACi-SC027 is prepared for the synthesis to label HDACi-SC027 with Gallium-67 or Gallium-68 radionuclide for producing imaging tracers for tracking the HDACi.

### BACKGROUND OF THE INVENTION

1. Field of the Invention

The present invention relates to nuclear medicine tracers and synthesis method thereof, in particular, to compounds of labeled histone deacetylase inhibitors used for diagnosing cancer and Alzheimer's disease by bonding with hyperactivity histone deacetylase in human body.

2. Description of the Related Art

Conventional studies about cancer and development dysplasia of organs like colon, rectum, cervix, stomach, and prostate have not been a satisfactory outcome. Besides, the research of natural aging indicates that cerebral atrophy is an early sign of neurodegeneration related to cognitive deficit and loss of memory, and dementia like Alzheimer's disease usually leads the patient of such disease to an unrecoverable situation for unknown causes and lacking of a measure of early diagnosis of Alzheimer's disease.

Early discovery, diagnosis and curing of cancer and Alzheimer's disease depend on diagnostic methods being used. According to research in cancer and development dysplasia of body organs, overexpression of histone deacetylase 2 (HDAC-2) does exist in both cases and in many cases of those diseases, such as colon, rectum, cervix, stomach, and prostate etc. Furthermore, the research in recent years also pointed out that chromatin modification in the brain cells is related to the memory formation which is influenced intensely by histone deacetylase, for example, mouse with abnormal secretion of histone deacetylase enzyme could lost part of memory as same as the symptom of Alzheimer's disease. Thus, dosing histone deacetylase inhibitors will be a new hope to the treatment of cancer and Alzheimer's disease.

Histone deacetylase inhibitors (HDAC inhibitors or HDACi) are medication for controlling histone deacetylase in the human body and being used to treat cancer and neurodegeneration in the medical research by medical industries. These specific mechanisms of inhibitor are disclosed in the characterization of genomics approaches presented in the literature, for example, Richon et al. found that HDAC inhibitors can control the tumor suppression function of tumor protein (P53) through introducing cyclin-dependent kinase inhibitor 1(P21 or WAF1).

HDAC inhibitors have been a hot spot of medication research as a targeted anti-tumor medication. The existing HDAC inhibitors are mainly divided into four categories according to formula, comprising: (a) hydroxamic acids, suchlike Vorinostat; (b) cyclic tetrapeptide, suchlike Romidepsin (FK228) and depsipeptide; (c) benzoylamide, suchlike MS-275 and SC-027; (d) short-chain fatty acid, suchlike valproic acid and butyrate. The efficacies of HDAC inhibitors for treating hematologic malignancies and solid tumors are confirmed both in vivo and vitro experiments. The vitro experiment confirmed that HDAC inhibitors exhibits good anti-tumor effect to the tumor cell of bladder, bone, breast, uterus, central nervous system, esophagus, lung, ovary, pancreas, or prostate by tumor cell apoptosis, proliferation inhibition and cell cycle arrest, and many types of HDAC inhibitors are entering phase I or II or III of clinical study for their multiple paths and high efficiencies for anticancer.

Vorinostat (suberoylanilide hydroxamic acid, SAHA) and Romidepsin (cyclic peptide) are approved to be listed for applying to cutaneous T-cell lymphoma (CTCL) and the application of the treatment of solid tumor is also in clinical trials. The benzoylamide HDAC inhibitors chidamide developed by Chipscreen Ltd. is approved by FDA for clinical research in USA to confirm that the new type HDAC inhibitors in small doses and low concentration can induce tumor cell differentiation and selective apoptosis for anti tumor proliferation and be non-toxic to normal cells.

Basic checks for diagnosis of Alzheimer's disease (AD) comprise neuropsychological testing, blood routine, biochemical test (liver and kidney function), concentration of vitamin B12 in blood, thyroid function, serological test for syphilis, brain computed tomography or magnetic resonance imaging, and so forth. The drugs used for positron high-end diagnostic imaging methods are known as F-18-AV45 and F-18-PIB based on amyloid hypothesis, and the drugs based on tau hypothesis are not listed at the moment. The image of diagnosis of Alzheimer's disease using F-18-AV45 and F-18-PIB could only determine if a patient is not suffering from Alzheimer's disease, but could not help determine if a patient is suffering from Alzheimer's disease or aging.

U.S. Pat. No. 7,803,800 discloses compounds of histone deacetylase having antitumor activity by promoting apoptosis and inhibiting cell proliferation on a panel of tumor cells. U.S. Pat. No. 8,716,285 discloses compounds having anti-proliferation and differentiation-inducing activity, which results in inhibition of tumor cell proliferation and induction of apoptosis. Even though the conventional compounds of the cited references could inhibit tumor cell proliferation, they could not be used to determine the location of tumor and diagnose the cause of disease.

### SUMMARY OF THE INVENTION

The present invention provides novel compounds for tracking histone deastylase inhibitors in vivo for the purpose of determining where the histone deastylase in vivo is having overexpression in order to make a good early diagnosis of tumor.

One object of the present invention is to provide synthesis methods of combining the HDAC inhibitor SC027

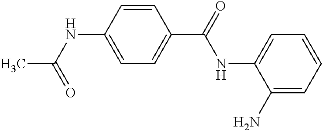 [SMILES: CC(=O)Nc1ccc(C(=O)Nc2ccccc2N)cc1]

(SC027's chemical name: 4-Acetylamino-N-(2′-aminophenyl) benzamide, commercial name: Acetyldinaline or Tacedinaline, empirical formula: C₁₅H₁₅N₃O₂, molecular mass:269.3, appearance: crystalline solid) with labeling radionuclide, such as Fluorine-18, Iodine-123, Iodine-125, Iodine-131, Carbon-11, Gallium-67, and Gallium-68, to produce a new type of tracer in nuclear medicine, such as

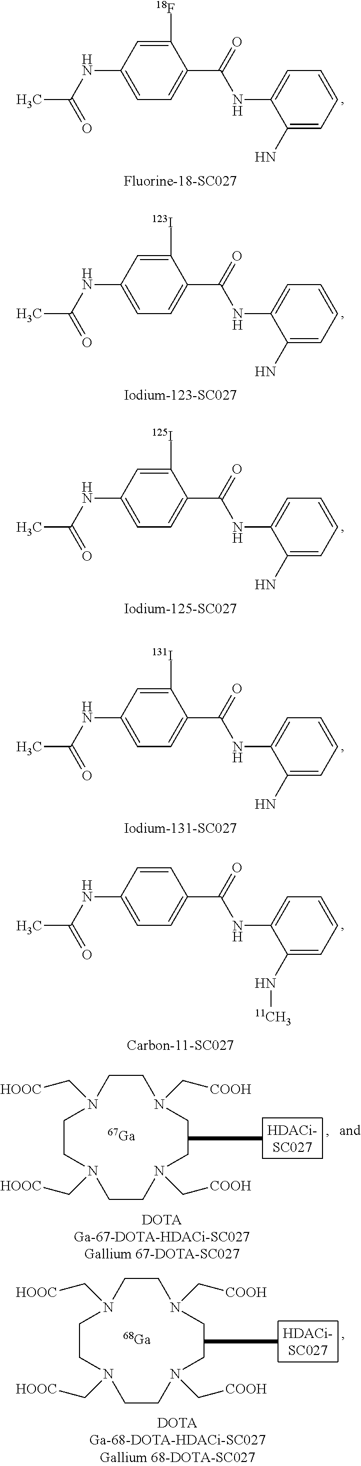

to be combined with the histone deacetylase having overexpression in vivo to obtain effective imaging agents of HDAC to be used in nuclear medicine.

Another object of the present invention is to provide compounds utilized for diagnosis of cancer and Alzheimer's disease by using the characteristic of positron decay of isotope, such as C-11, F-18, Ga-68 and so forth, to cause an annihilation reaction that makes positrons released from positron decay encountering electrons of cells to form a pair of opposed directions gamma rays for gaining images through positron emission tomography (PET).

Still another object of the present invention is to provide a synthesis method for combining HDAC inhibitor SC027 with labeling radionuclides Ga-67 or Ga-68 by a required labeling precursor DOTA-SC027.

Further object of the present invention is to provide a labeling precursor DOTA-SC027 which is a compound of DOTA-NHS-ester

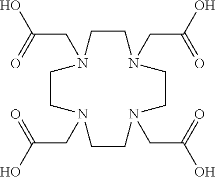 [SMILES: O=C(O)CN1CCN(CC(=O)O)CCN(CC(=O)O)CCN(CC(=O)O)CC1]

(chemical name: 2,2′,2″-(10-(2-((2,5-dioxopyrrolidin-1-yl)oxy)-2-oxoethyl)-1,4,7,10-tetraaza cyclododecane-1,4,7-triyl)triacetic acid) and HDACi-SC027 to produce the imaging agent of HDAC inhibitors.

### DETAILED DESCRIPTION OF THE PREFERRED EMBODIMENTS

To achieve the objects listed above, in an embodiment, the present invention provides a synthesis method of labeling precursor DOTA-SC027

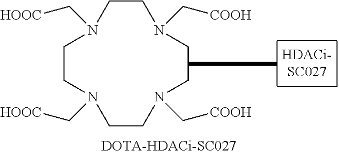 [SMILES: O=C(O)CN1CCN(CC(=O)O)CCN(CC(=O)O)CCN(CC(=O)O)CC1]

comprising steps of:

Step 1) putting DOTA-NHS-ester (N-hydroxysuccinimide, NHS) and SC027 into two glass bottles separately;
Step 2) dropping dimethylformamide (DMF) into the bottles of DOTA and SC027 separately by trickling around in those glass bottles to dissolve dimethylformamide completely;
Step 3) fixing a round-bottom flask on a mixer and adding triethylamine (TEA) into the glass bottle of DOTA by micropipette, then moving the solution of DOTA in the glass bottle to the round-bottom flask by micropipette;
Step 4) adjusting the speed of the mixer to about 500 rpm, dropping the solution of SC027 into the round-bottom flask by micropipette after mixing for one hour and keep the solution react for about 24 hours;
Step 5) proceeding a freeze-drying process after the solution of SC027 reacting for about 24 hours; and
Step 6) obtaining a finished compound of labeled precursor DOTA-NHS-HDACi-SC027.

In one embodiment, the present invention provides a method of synthesizing a labeled precursor DOTA-NHS-ester-HDACi-SC027 comprising steps of:

Step 1) placing a weighing paper on an electronic scale to zero after calibrating the electronic scale;
Step 2) weighing DOTA-NHS-ester about 165.5 mg (the actual measurement is 165.84 mg) and HDACi-SC027 about 106.6 mg (the actual measurement is 106.93 mg);
Step 3) putting the weighted DOTA-NHS-ester and HDACi-SC027 into two glass bottles separately and sealing up the remaining chemical agents with parafilm;
Step 4) dropping 1 ml of dimethylformamide into the DOTA and another 1 ml of dimethylformamide into the HDACi-SC027 with trickling around in the two glass bottles by micropipette for completely dissolving or using bottle closures to seal up those glass bottles and shaking them for dissolving the agents in those bottles completely;
Step 5) washing a 25 ml round-bottom flask with acetone and drying the round-bottom flask;
Step 6) fixing the round-bottom flask on a heating mixer;
Step 7) adding about 4.34 ul of triethylamine into the DOTA solution in the glass bottle and moving the DOTA solution to the round-bottom flask by micropipette;
Step 8) starting the heating mixer to about speed 500 rpm and an exhaust fan;
Step 9) after mixing the DOTA solution for about one hour, adding the HDACi-SC027 solution into the DOTA solution for heating and mixing at speed about 500 rpm for about 24 hours;
Step 10) after mixing and heating for about 24 hours, observing the color of DOTA-NHS-ester-HDACi-SC027 solution in the heating mixer remaining yellow and turning off the heating mixer and dispensing the DOTA-NHS-ester-HDACi-SC027 solution into two little bottles being 0.5 ml and 1.6 ml, respectively;
Step 11) placing the two bottles with air gap and filled with the DOTA-NHS-ester-HDACi-SC027 solution on an iron plate and cooling the DOTA-NHS-ester-HDACi-SC027 solution by pouring liquid nitrogen on the iron plate for vacuuming with a freeze dryer, dissolving back the dimethylformamide in the DOTA-NHS-ester-HDACi-SC027 solution for analyzing by high performance liquid chromatography (HPLC), and using the remaining DOTA-NHS-ester-HDACi-SC027 solution preserved in a refrigerator at temperature about 20 degree Celsius(° C.) for subsequent labeling process with radionuclide of Ga-67 or Ga-68.

In another embodiment, the present invention also provides a method of synthesizing HDACi-SC027 labeled with Gallium 67 or Gallium 68 to produce a new nuclear medicine tracer of Ga67-DOTA-SC027 or Ga68-DOTA-SC027 comprising two ways of:

method 1) putting Gallium 67 or Gallium 68 into DOTA-SC027 and heating for about 15˜30 minutes at temperature range from 85 to 100 degree Celsius; or
method 2) dispensing DOTA-SC027 solution into little glass bottle kits for freeze-drying process, after filling inert gas into the kits and sealing up the kits, injecting Gallium 67 or Gallium 68 with pH buffer into the kits for dissolving by shaking the kits and placing the kits in an autoclave for about 15˜30 minutes at temperature range from 85 to 100 degree Celsius.

There are two chemical formulas can be obtained from the methods of the present invention including:

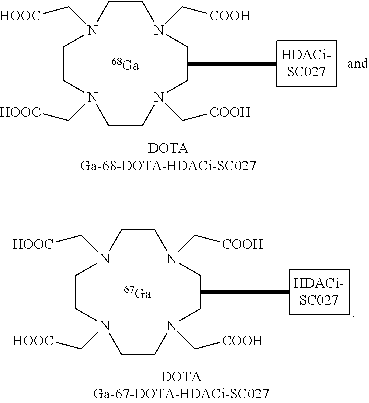

What is claimed is:

1. An imaging compound used as a tracer of histone deastylase inhibitors for diagnosis of tumors in nuclear medicine, wherein the compound is synthesized by labeling radionuclide and histone deastylase inhibitor SC027 having chemical formula: 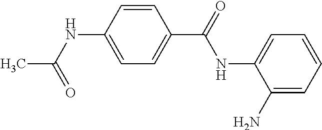 [SMILES: CC(=O)Nc1ccc(C(=O)Nc2ccccc2N)cc1]
chemical name: 4-Acetylamino-N-(2′-aminophenyl)benzamide, commercial name: Acetyldinaline or Tacedinaline, and empirical formula: C₁₅H₁₅N₃O₂.

2. An imaging compound according to claim 1, wherein the compound comprises:

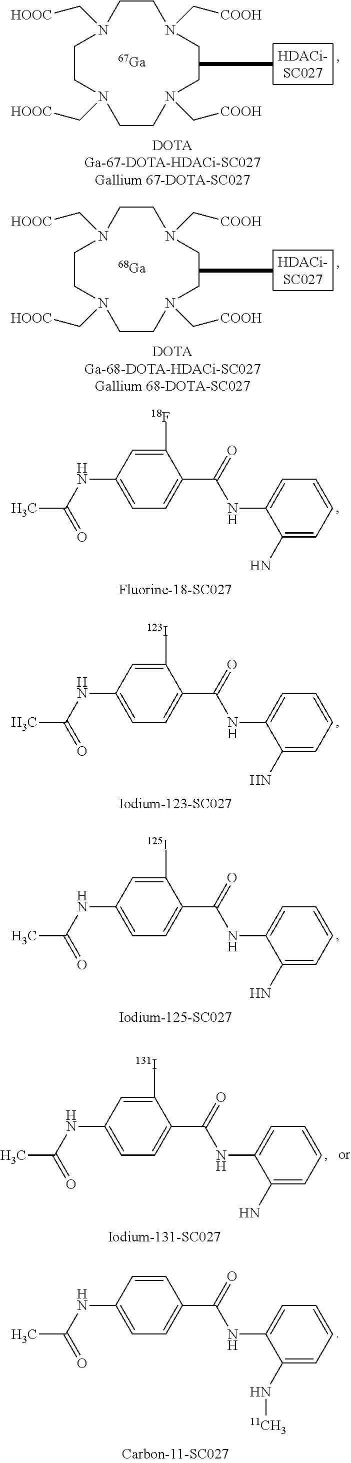

3. An imaging compound according to claim 2, wherein the compound requires a labeling precursor DOTA-SC027 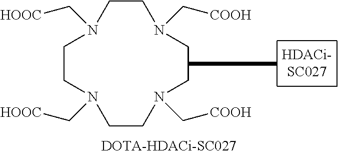 [SMILES: O=C(O)CN1CCN(CC(=O)O)CCN(CC(=O)O)CCN(CC(=O)O)CC1]
which is synthesized by a method comprising steps of:
Step 1) putting DOTA-NHS-ester (N-hydroxysuccinimide, NHS) 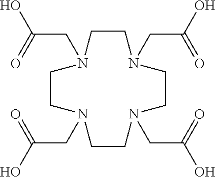 [SMILES: O=C(O)CN1CCN(CC(=O)O)CCN(CC(=O)O)CCN(CC(=O)O)CC1]
and histone deastylase inhibitor SC027 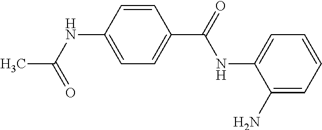 [SMILES: CC(=O)Nc1ccc(C(=O)Nc2ccccc2N)cc1]
into two glass bottles separately;
Step 2) dropping dimethylformamide (DMF) into the bottles of DOTA and SC027 separately by trickling around in those glass bottles to dissolve dimethylformamide completely;
Step 3) fixing a round-bottom flask on a mixer and adding triethylamine (TEA) into the glass bottle of DOTA by micropipette, then moving the solution of DOTA in the glass bottle to the round-bottom flask by micropipette;
Step 4) adjusting the speed of the mixer to about 500 rpm, dropping the solution of SC027 into the round-bottom flask by micropipette after mixing for about one hour and keep the solution react for about 24 hours;
Step 5) proceeding a freeze-drying process after the solution of SC027 reacting for about 24 hours; and
Step 6) obtaining a finished compound of labeled precursor DOTA-NHS-HDACi-SC027.

4. An imaging compound according to claim 2, wherein the method of synthesizing Gallium-67-DOTA-SC027 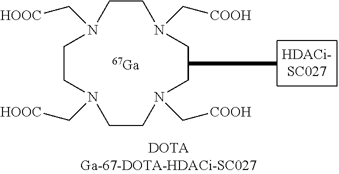
or Gallium-68-DOTA-SC027 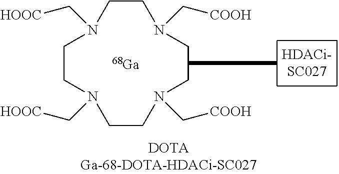
by combining labeling radionuclide Gallium 67 or Gallium 68 with histone deastylase inhibitor SC027 comprises two methods of:
method 1) putting Gallium 67 or Gallium 68 into DOTA-SC027 and heating for about 15˜30 minutes at 85˜100 degree Celsius; or
method 2) dispensing DOTA-SC027 solution into little glass bottle kits for freeze-drying process, after filling inert gas into the kits and sealing up the kits, injecting Gallium 67 or Gallium 68 with pH buffer into the kits for dissolving by shaking the kits and placing the kits in an autoclave for about 15˜30 minutes at about 85˜100 degree Celsius.

5. An imaging compound according to claim 2, wherein the Fluorine-18-SC027 is synthesized by histone deastylase inhibitor SC027 and labeling radionuclide Fluorine-18 in chemical formula of

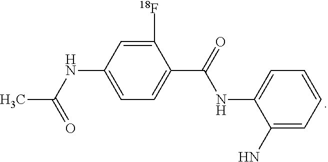 [SMILES: CC(=O)Nc1ccc(C(=O)Nc2ccccc2N)c([18F])c1]

6. An imaging compound according to claim 2, wherein the Iodium-123-SC027, Iodium-125-SC027, or Iodium-131-SC027 is synthesized by histone deastylase inhibitor SC027 and labeling radionuclide Iodium-123, Iodium-125, Iodium-131 with chemical formula of, respectively,

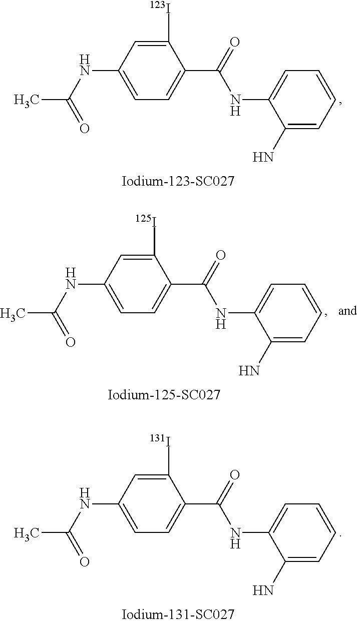 [SMILES: CC(=O)Nc1ccc(C(=O)Nc2ccccc2N)c([123I])c1 ; CC(=O)Nc1ccc(C(=O)Nc2ccccc2N)c([125I])c1 ; CC(=O)Nc1ccc(C(=O)Nc2ccccc2N)c([131I])c1]

7. An imaging compound according to claim 2, wherein the Carbon-11-SC027 is synthesized by histone deastylase inhibitor SC027 and labeling radionuclide Carbon-11 in chemical formula of 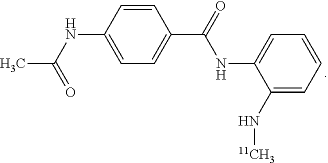 [SMILES: CC(=O)Nc1ccc(C(=O)Nc2ccccc2N[11CH3])cc1]
